(19) United States
(12) Patent Application Publication
(10) Pub. No.: US 2025/0000181 A1
(43) Pub. Date: Jan. 2, 2025

(54) UTILITY PANTS

(71) Applicant: Infection Control Products LLC, Dallas, TX (US)

(72) Inventor: Joseph A. Graneto, III, Dallas, TX (US)

(21) Appl. No.: 18/341,820

(22) Filed: Jun. 27, 2023

(51) Int. Cl. A41D 1/06; A41D 15/00; A41D 27/08; A41D 27/20; A61F 5/02

(52) U.S. Cl. CPC A41D 1/06; A41D 13/01; A41D 15/002; A41D 27/201; A61F 5/028; A41D 2200/10; A41D 2300/322

(57) ABSTRACT

The utility pants may include a support belt, an upper portion attached to the support belt, and a lower portion configured to attach to the upper portion. The upper portion and the lower portion may be attached via a plurality of zippers. When the upper portion and the lower portion are attached, the utility pants may be longer than standard shorts. When the upper portion and the lower portion are removed from one another, the utility pants may be similar to standard shorts. The utility pants may also include a plurality of lights, reflective tape, and/or a plurality of pockets. The lights, reflective tape, and pockets may be configured to be attached to at least one of the upper portion and the lower portion.

### CROSS-REFERENCE TO RELATED APPLICATIONS

None.

### FIELD OF INVENTION

The present invention relates generally to utility pants. More specifically, the invention relates to a pair of utility pants that may provide a support belt, lights, reflective tape, and/or removable lower portions.

### BACKGROUND OF INVENTION

Members of various industries (e.g., delivery drivers) face issues with carrying several different components while performing deliveries. For example, delivery drivers may need to lift heavy objects, and as such, delivery drivers may carry support belts with them. Such support belts may provide support to users' backs when lifting heavy objects. Additionally, for example, delivery drivers may need to deliver packages when it is dark outside. Delivery drivers may carry flashlights in order to see in the dark.

Additionally, many delivery drivers may be concerned with crossing streets. As such, delivery drivers may bring reflective vests with them in order to address these concerns. Delivery drivers may also deliver packages at all times of year and in all different climates. At times, shorts may be desired, while at other times, pants are desired. Due to this, delivery drivers may carry different pairs of garments to wear depending on the climate.

Although each of these different components are useful, delivery drivers must remember to pack each of the components and may handle all of them at different times. This may be difficult as the delivery drivers must then manage several devices while performing an already-busy task.

### SUMMARY OF INVENTION

The present invention overcomes many of the shortcomings and limitations of the prior art devices discussed above. The invention described herein includes several embodiments of utility pants. The utility pants may be used by anyone, but for exemplary purposes, delivery drivers may use the utility pants.

The present invention solves the issues caused by existing systems by providing users with an all-in-one device configured to provide each of the desired components as discussed above. The utility pants are configured to provide the support belt, lights, reflective tape, and/or removable lower portions. This prevents the user from needing to carry, manage, and handle each of the individual components.

The utility pants may include a support belt, an upper portion attached to the support belt, and a lower portion configured to attach to the upper portion. The upper portion and the lower portion may be attached via a plurality of zippers. When the upper portion and the lower portion are attached to one another, the utility pants may be the length of standard “pants.” When the upper portion and the lower portion are removed from one another, the utility pants may be similar in length to standard “shorts.” The utility pants may also include a plurality of lights, reflective tape, and/or a plurality of pockets. The lights, reflective tape, and pockets may be configured to be attached to at least one of the upper portion and the lower portion.

### BRIEF DESCRIPTION OF DRAWINGS

FIG. 1 is a front perspective view of utility pants according to one embodiment of the present invention;

FIG. 2 is a rear perspective view of the utility pants of FIG. 1; and

FIG. 3 is a front perspective view of utility pants according to one embodiment of the present invention.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

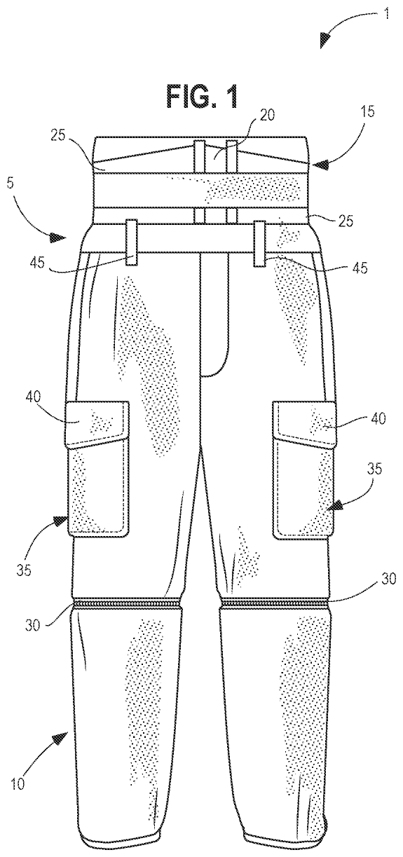

Turning first to FIG. 1, the utility pants 1 may be made of any suitable material, including cotton, polyester, spandex, etc. (including any combination of the foregoing fabrics). The utility pants 1 may be reinforced with a waterproof coating and/or may be made of waterproof fabric. According to various embodiments, the utility pants 1 are durable and include reinforced stitching.

The utility pants 1 may include an upper portion 5 and a lower portion 10. The upper portion 5 may include a support belt 15. The support belt 15 may be integrally formed to the upper portion 5, such that the utility pants 1 include the upper portion 5 and support belt 15 at all times. The support belt 15 may be attached to the upper portion 5 in any manner known in the art. In one embodiment, the support belt 15 is sewn into the upper portion 5. In one embodiment, the support belt 15 is manufactured as a single piece with the upper portion 5. The support belt 15 may be elastic, such that the belt 15 may be used on all individuals, no matter their size or shape. In various embodiments, the support belt 15 is adjustable to fit various users. The support belt 15 may provide a user with support when lifting objects. The support belt 15 may be any belt known in the art to provide support to a user's lower back. In one embodiment, the support belt 15 may include a center portion 20 and an outer portion 25. The width of the center portion 20, which may be on the respective front and back of the utility pants 1, may be greater than the width of the outer portion 25, which may be located on the left and right side of the utility pants 1. Such differences in the widths may be conducive to providing back support.

The upper portion 5 may attach to the lower portion 10 using any manner known in the art, including via zippers 30. When the upper portion 5 is removed from the lower portion 10, the utility pants 1 may be configured to be shorts and may be shorter than standard pants. The lower portion 10 may be an additional piece of fabric that may attach to the upper portion 5 to create pants. The utility pants 1 may include lights (not illustrated). The lights may be configured to turn on as desired by a user.

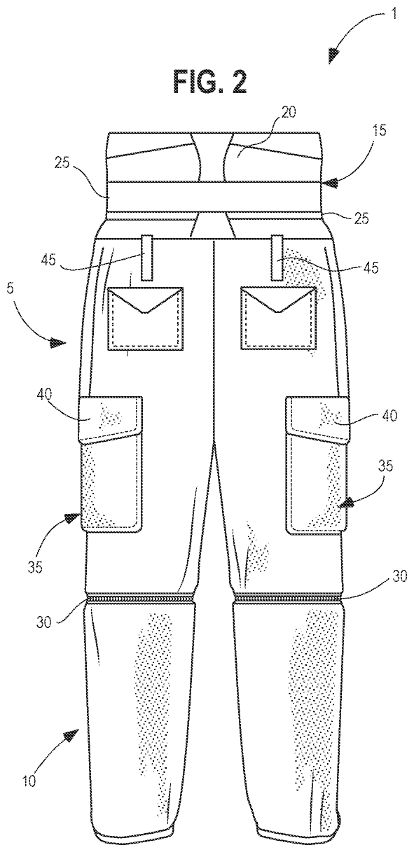

The upper portion 5 and lower portion 10 may include pockets 35 and other components useful for a standard pair of pants. As illustrated in FIG. 2, the upper portion 5 may include additional pockets 35. The pockets 35 may include covers 40, which may be configured to close via Velcro, buttons, or any other suitable mechanism. The pockets 35 may be sewn onto the utility pants 1. In various embodiments, the pockets 35 are formed within the utility pants 1. In one embodiment, the pockets 35 do not include covers 40. According to one embodiment, the utility pants 1 include belt loops 45 which may be configured to receive a standard belt used to tighten the waist of the utility pants 1.

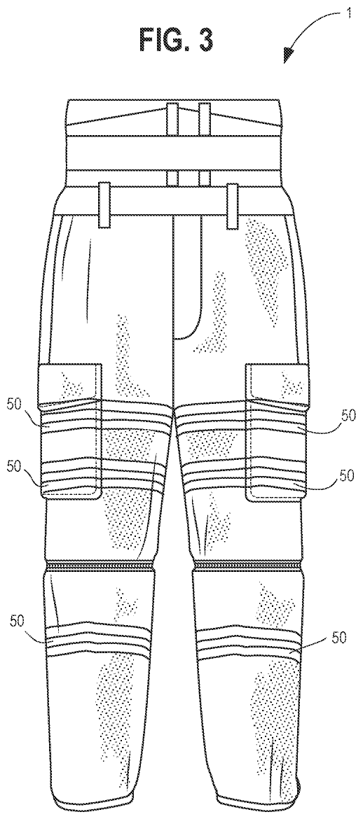

Turning to FIG. 3, the utility pants 1 may include reflective tape 50. According to various embodiments, the reflective tape 50 is wrapped around the legs of the utility pants 1 to form rings. The reflective tape 50 may be attached at a variety of different locations and in a variety of different manners on the utility pants 1. The reflective tape 50 may be attached to the utility pants 1, or in one embodiment, may be integrally formed with the utility pants 1. The reflective tape 50 may be placed along the utility pants 1. According to various embodiments, the reflective tape 50 is configured to reflect light that is directed toward the reflective tape 50. When reflecting, the individuals wearing the utility pants 1 may be seen by passersby, which may reduce risk when crossing streets, interacting with other individuals, etc.

As is evident from the foregoing description, certain aspects of the present invention are not limited by the particular details of the examples illustrated herein, and it is therefore contemplated that other modifications, applications, variations, or equivalents thereof, will occur to those skilled in the art. Many such changes, modifications, variations, and other uses and applications of the present constructions will, however, become apparent to those skilled in the art after considering the specification and the accompanying drawings. In addition, unless mention was made above to the contrary, it should be noted that all of the accompanying drawings are not to scale. All such changes, modifications, variations, and other uses and applications which do not depart from the spirit and scope of the present inventions are deemed to be covered by the inventions which are limited only by the claims which follow.

1. A pair of utility pants comprising:
a support belt;
an upper portion attached to the support belt;
a lower portion configured to attach to the upper portion; and
a plurality of pockets attached to at least one of the upper portion and the lower portion.

2. The pair of utility pants of claim 1, further comprising reflective tape attached to at least one of the upper portion and the lower portion.

3. The pair of utility pants of claim 1, wherein when the upper portion is removed from the lower portion, a length of the pair of utility pants is shortened.

4. The pair of utility pants of claim 1, wherein the upper portion and the lower portion are attached to one another via a plurality of zippers.

5. The pair of utility pants of claim 1, wherein the pair of utility pants includes at least one of cotton, polyester, and spandex.

6. The pair of utility pants of claim 1, wherein the support belt is elastic.

7. The pair of utility pants of claim 1, further comprising a plurality of lights attached to at least one of the upper portion and the lower portion.

8. The pair of utility pants of claim 1, wherein the plurality of pockets is covered via a plurality of pocket covers.

9. A pair of utility pants comprising:
a support belt;
an upper portion attached to the support belt;
a lower portion configured to attach to the upper portion; and
reflective tape attached to at least one of the upper portion and the lower portion.

10. The pair of utility pants of claim 9, a plurality of pockets attached to at least one of the upper portion and the lower portion.

11. The pair of utility pants of claim 9, wherein when the upper portion is removed from the lower portion, a length of the pair of utility pants is shortened.

12. The pair of utility pants of claim 9, wherein the upper portion and the lower portion are attached to one another via a plurality of zippers.

13. The pair of utility pants of claim 9, wherein the pair of utility pants includes at least one of cotton, polyester, and spandex.

14. The pair of utility pants of claim 9, wherein the support belt is elastic.

15. The pair of utility pants of claim 9, further comprising a plurality of lights attached to at least one of the upper portion and the lower portion.

16. The pair of utility pants of claim 9, wherein the reflective tape is wrapped around legs of the pair of utility pants to form a plurality of rings of reflective tape.

17. A pair of utility pants comprising:
a support belt;
an upper portion attached to the support belt; and
a lower portion configured to attach to the upper portion.

18. The pair of utility pants of claim 17, further comprising reflective tape attached to at least one of the upper portion and the lower portion.

19. The pair of utility pants of claim 17, further comprising a plurality of pockets attached to at least one of the upper portion and the lower portion.

20. The pair of utility pants of claim 17, further comprising a plurality of lights attached to at least one of the upper portion and the lower portion.
